(19) United States
(12) Patent Application Publication
(10) Pub. No.: US 2026/0061145 A1
(43) Pub. Date: Mar. 5, 2026

(54) Carbon-dioxide and Oxygen Respiratory Ventilator Energy Tracker (CORVET)

(71) Applicant: Massachusetts Institute of Technology, Cambridge, MA (US)

(72) Inventors: Gary A Shaw, Hartwell, GA (US); Lawrence M Candell, Arlington, MA (US); Kyle THOMPSON, Boulder, CO (US); Allison Norloff, Arlington, MA (US); Donald Johnson, Woburn, MA (US); Roberto Buizza, Malden, MA (US); Francesco Vicario, Boston, MA (US)

(73) Assignees: Massachusetts Institute of Technology, Cambridge, MA (US); KONINKLIJKE PHILIPS N.V., EINDHOVEN (NL)

(21) Appl. No.: 18/405,392

(22) Filed: Jan. 5, 2024

Related U.S. Application Data
(60) Provisional application No. 63/488,915, filed on Mar. 7, 2023.

(51) Int. Cl. A61M 16/00; A61M 16/08; A61M 39/22

(52) U.S. Cl. CPC A61M 16/024; A61M 16/0003; A61M 16/0875; A61M 39/223; A61M 2016/0042; A61M 2202/0007; A61M 2202/0208; A61M 2205/3327; A61M 2205/3334; A61M 2205/3368; A61M 2205/3379; A61M 2205/50; A61M 2230/42

(57) ABSTRACT

A respiratory monitoring system with improved detection of oxygen consumption is described. The system uses one or two mixing chambers and samples gases at selective locations for reliable detection of oxygen over extended periods of time with autonomous detection of calibration drift. The system can be used for indirect calorimetry and monitoring health of a subject.

### CROSS-REFERENCE TO RELATED APPLICATION(S)

The present application claims the priority benefit, under 35 U.S.C. 119(e), of U.S. Application No. 63/488,915, filed Mar. 7, 2023, which is incorporated herein by reference in its entirety for all purposes.

### GOVERNMENT SUPPORT

This invention was made with government support under FA8702-15-D-0001 awarded by the U.S. Air Force. The government has certain rights in the invention.

### BACKGROUND

Continuous measurement of the volume of oxygen consumed (VO₂) by a person or animal and the volume of carbon dioxide produced (VCO₂) enables tracking of energy expenditure, macronutrient utilization, and metabolic health metrics. There are currently no commercially-available systems capable of providing continuous, unattended multi-day metabolic tracking of ventilated patients to provide highly accurate estimates of macronutrient utilization and energy expenditure. Although a number of commercial indirect calorimetry systems exist, studies have shown them to be too time consuming, costly, and inaccurate for clinical use. A 2012 study showed that the parametric formulas used to estimate required nutritional needs of ventilated patients achieve accurate measurements to within 10% or better accuracy less than 25% of the time, resulting in overfeeding in 27.4% of the patients and underfeeding in 48.3% of the patients. (See, E. De Waele, H. Spapen, P. M. Honore, S. Mattens, T. Rose and L. Huyghens, “Bedside calculation of energy expenditure does not guarantee adequate caloric prescription in long-term mechanically ventilated critically ill patients: a quality control study,” The Scientific World Journal, 2012.) Numerous studies have shown that both underfeeding and overfeeding of mechanically ventilated patients negatively impacts both recovery time and mortality.

Traditionally, expensive and bulky metabolic carts having multi-liter mixing chambers are used to measure VO₂. These instruments require a long start-up time and are used intermittently. More compact systems, referred to as breath-by-breath systems, rely on high-frequency side-stream measurements of O₂ concentration (typically requiring expensive O₂ sensors). Their accuracy is highly dependent on the precise time alignment of the sequential measurements of O₂ and CO₂ concentrations and gas flow differential waveforms, in all ventilation and measurement conditions. Such breath-by-breath systems are not suitable for use with mechanically ventilated patients. A system designed for use with mechanically ventilated patients and employing a miniature mixing chamber has been previously developed, but requires manual calibration before each use, and is intended only for short-term measurements (on the order of 30 minutes) before clogging of the systems filter and sample lines inhibits measurement accuracy. A short 30-minute measure of energy expenditure once or even twice a day, while beneficial, is far too infrequent to produce a reliable estimate of 24-hour energy expenditure and macronutrient utilization. Furthermore, to date, the only available miniature mixing chamber metabolic cart requires disrupting patient ventilation to insert the sampling apparatus into the breathing line of the patient.

### SUMMARY

The present disclosure relates to systems and methods for long-term continuous measurement of respiratory volume of oxygen consumed (VO₂) and carbon dioxide produced (VCO₂) by a person or animal with monitoring of calibration stability and techniques for cancelling drift to improve measurement accuracy without disruption of the patient ventilation. The monitored VO₂ and VCO₂ amounts can be used to estimate the energy expenditure (e.g., calories consumed over time), macronutrient utilization, and other values relevant to health of a subject (such as a patient in a clinical setting). Accuracy of the system is improved by using one or two mixing chambers (for inhalation gas and for exhaled gas) which allow sampled gases to mix over several respiratory cycles, effectively performing a mathematical average of gas concentrations. Additionally, gas sampling circuits for the mixing chamber(s) are designed to sample at least the exhaled gas at a location where the gas is well mixed within its flow circuit. With such improvements, the T₉₀ time constant associated with the VO₂ and VCO₂ sensors can be reduced, decreasing their cost.

Some implementations relate to a respiratory monitoring system comprising a first multi-way valve, a mixing chamber having an input port fluidically coupled to the first multi-way valve, and an oxygen sensor coupled to the mixing chamber. The respiratory monitoring system can further include an inhalation input-sampling flow line fluidically coupled at a first end to the first multi-way valve and to fluidically couple at a second end to an inhalation line that is fluidically coupled to breathing apparatus for a subject. The respiratory monitoring system can further include an exhalation input-sampling flow line fluidically coupled at a first end to the first multi-way valve and to fluidically couple at a second end to an exhalation line that is fluidically coupled to the breathing apparatus for the subject. The first multi-way valve can be configured to switch coupling of the input port of the mixing chamber between a first configuration where the input port is fluidically coupled to the inhalation input-sampling flow line and a second configuration where input port is fluidically coupled to the exhalation input-sampling flow line.

Some implementations relate to a method of respiratory monitoring with a respiratory monitoring system. The method can include acts of: during a first interval of time, receiving inhalation gas for a subject in a mixing chamber, wherein the inhalation gas is sampled from an inhalation line of the respiratory monitoring system and the inhalation line connects to breathing apparatus (e.g., a mechanical ventilator) used by the subject; detecting a first concentration of oxygen (and optionally a first concentration of carbon dioxide) in the mixing chamber with an oxygen sensor during the first interval of time; and changing, with a multi-way valve, coupling of an input port of the mixing chamber from being fluidically coupled to the inhalation line to being fluidically coupled to an exhalation line of the respiratory monitoring system. The method can further include, during a second interval of time, receiving exhalation gas from the subject in the mixing chamber, wherein the exhalation gas is sampled from the exhalation line and the exhalation line is coupled to the breathing apparatus; detecting a second concentration of oxygen (and optionally a second concentration of carbon dioxide) in the mixing chamber with the oxygen sensor during the second interval of time; and computing a value relevant to health of the subject based at least in part on a difference between the first and second concentrations of oxygen (and optionally on a difference between the first and second concentrations of carbon dioxide).

All combinations of the foregoing concepts and additional concepts discussed in greater detail below (provided such concepts are not mutually inconsistent) are part of the inventive subject matter disclosed herein. In particular, all combinations of subject matter appearing in this disclosure are part of the inventive subject matter disclosed herein. The terminology used herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1 depicts a block diagram of a respiratory monitoring system.

FIG. 2 depicts further details of a respiratory monitoring system with two mixing chambers that can be swapped between inhalation and exhalation lines.

FIG. 3A depicts an example of a passive flow divider that can be used in the system of FIG. 2 to produce a rate-proportional, side-stream sample.

FIG. 3B depicts further details of the passive flow divider of FIG. 3A.

FIG. 4A depicts the respiratory monitoring system of FIG. 2 operating with a first valve configuration.

FIG. 4B depicts the respiratory monitoring system of FIG. 2 operating with a second valve configuration that swaps the mixing chambers between inhalation and exhalation lines.

FIG. 5 depicts an implementation for a respiratory monitoring system in a clinical setting where a subject is ventilated by a mechanical ventilator.

FIG. 6A and FIG. 6B are block diagrams for an implementation of a respiratory monitoring system that uses a single mixing chamber.

FIG. 6C illustrates further details of the respiratory monitoring system having a single mixing chamber in the configuration of FIG. 6A.

FIG. 6D illustrates further details of the respiratory monitoring system having a single mixing chamber in the configuration of FIG. 6B.

### DETAILED DESCRIPTION

### 1. Overview

Measurements of the volume of oxygen consumed (VO₂) and the volume of carbon dioxide produced (VCO₂) by a patient per unit of time are highly desirable in several medical applications, including anaesthesia, critical care, and nutrition management. Often, simultaneous measurements of VO₂ and VCO₂ are referred to as metabolic monitoring since the amount of energy expenditure by the body can be inferred by these two measurements (a technique known as indirect calorimetry). Furthermore, VO₂ measurements are of particular interest in critical patients since VO₂ is intimately related to the transport of oxygen from the lungs to the tissues that need it. As such, VO₂ provides significant insights into the cardiovascular and metabolic status of a patient.

For purposes of this disclosure, VO₂ is defined as the difference between the inhaled volume of O₂ and the exhaled volume of O₂ per unit of time:

$${VO}_{2} = {V}_{inh} {O}_{2} - {V}_{exh} {O}_{2} \qquad ( 1 )$$

The inhaled volume Vᵢₙₕ O₂ and exhaled volume Vₑₓₕ O₂ are determined by direct measurement or indirect estimation to compute VO₂. The rate of O₂ consumption is typically determined from the subject's respiratory rate. Typically, VO₂ is expressed in units of litres per minute (LPM).

Similarly, VCO₂ is defined as the difference between the exhaled volume of CO₂ and the inhaled volume of CO₂ per unit of time (e.g., also expressed in units of LPM):

$${VCO}_{2} = {V}_{inh} {CO}_{2} - {V}_{exh} {CO}_{2} \qquad ( 2 )$$

For carbon dioxide, the inhaled volume Vᵢₙₕ CO₂ can normally be assumed to be equal to zero when tanked gases are supplied or a small amount that is precomputed based on the known, small concentration of CO₂ in ambient air, typically 300 to 400 PPM.

Although systems and methods for continuous VCO₂ monitoring in mechanically ventilated patients are well-established, VO₂ measurements are far from ubiquitous in the intensive care unit of clinical settings. This difference is due in part to the comparative ease of monitoring CO₂ production. Since the amount of CO₂ in ambient air is so small, the relative change in CO₂ concentration produced by a human in an exhaled breath is relatively easy to detect (e.g., changing from about 0.04% to about 4.4% (an increase in volume concentration by a factor of more than 100×) with each respiratory cycle). The considerably smaller relative change in O₂ is harder to detect accurately on time scales (several breaths) over which it can change when consumed by a human (e.g., for an ambient air inspired O₂ concentration of nominally 20.4%, 4.4% O₂ consumption represents a reduction in concentration by only a factor of 1.275, and for the case of a ventilator supplying 100% O₂, the reduction in concentration is only a factor of 1.04). The implication is that the O₂ sensor should span a greater range while maintaining the same absolute concentration error achieved by the CO₂ sensor. Accordingly, the size, cost, and accuracy of existing systems have limited their application of VO₂ measurements.

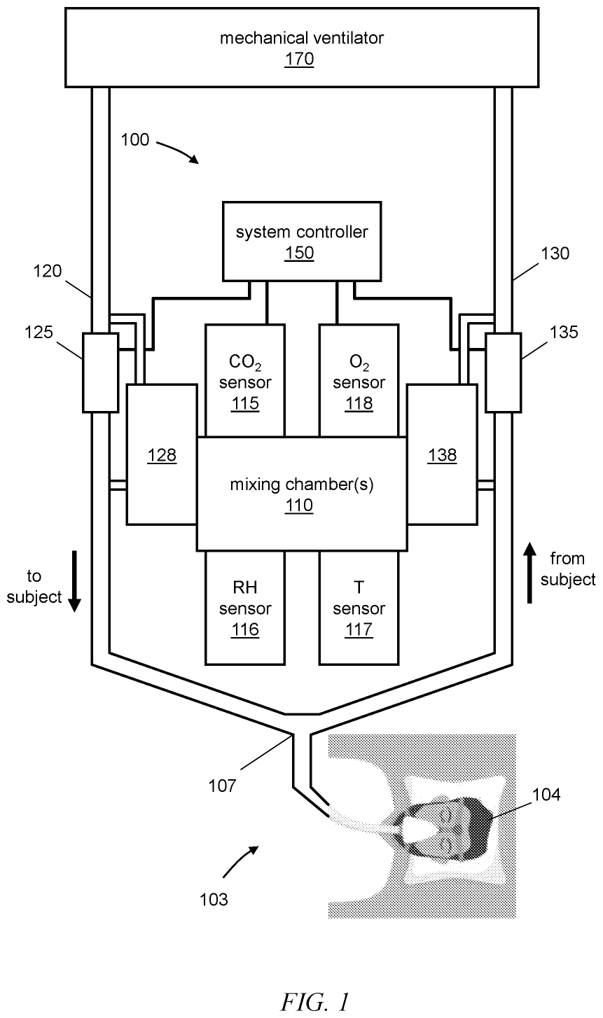

FIG. 1 depicts a block diagram of a respiratory monitoring system 100 that can measure VO₂ with improved accuracy for a patient. The respiratory monitoring system 100 does not require fast gas sensors to perform breath-by-breath measurements and does not require large mixing chambers to capture the entire volume of each exhalation for multiple breaths. The respiratory monitoring system 100 can also measure VCO₂ with high accuracy. The system 100 includes at least one mixing chamber 110, at least one CO₂ sensor 115 coupled to the mixing chamber, at least one O₂ sensor 118 coupled to the mixing chamber, at least one relative humidity sensor 116 coupled to the mixing chamber, at least one temperature sensor 117 coupled to the mixing chamber, a first flow sensor 125, a second flow sensor 135, and a system controller 150.

The mixing chamber(s) 110 is (are) in fluid communication with an inhalation line 120 and with an exhalation line 130. The inhalation line 120 can connect at one end to a source of breathable gas and connect at its other end to breathing apparatus 103 for a subject 104 (e.g., a patient). The breathing apparatus 103 may or may not include a Y-piece 107 than can include a valve to direct exhalations to the exhalation line 130. The exhalation line 130 can connect at one end to a reservoir or open space and connect at its other end to the breathing apparatus 103 for the subject 104. In some implementations, the air supply source can be a mechanical ventilator 170, which may also contain a reservoir or port for exhaled gas from a subject 104. Other breathing instruments (e.g., an oxygen supply, a continuous positive airway pressure machine, a controlled ambient air source, etc.) may be used instead of the ventilator 170 as the air supply source in some applications.

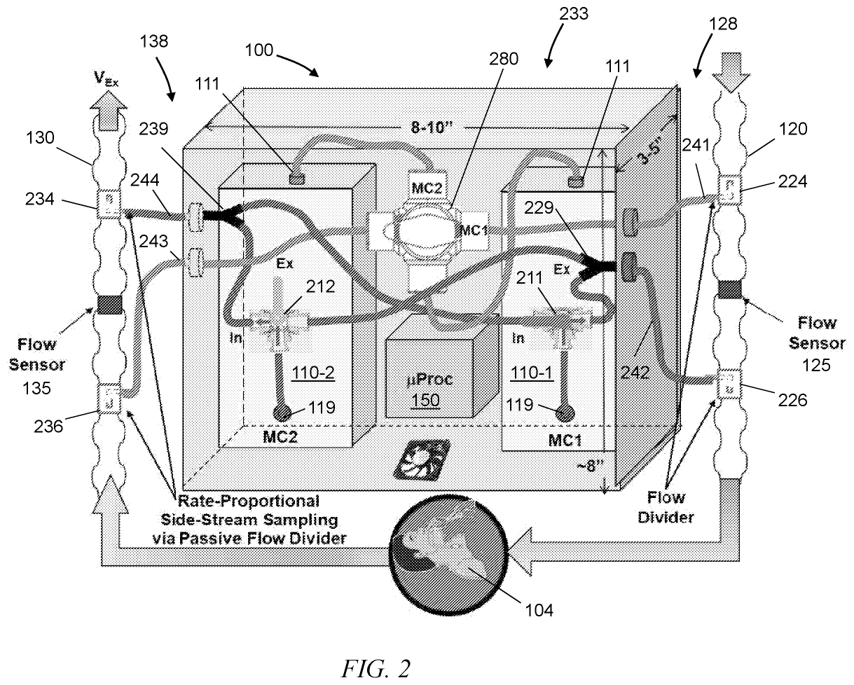

The mixing chamber(s) can couple to the inhalation line 120 via an inhalation flow circuit 128 and can also couple to the exhalation line 130 via an exhalation flow circuit 138. These flow circuits can include multi-way valves and flow line couplers, as described in further detail below in connection with FIG. 2. The couplings to the inhalation line 120 and exhalation line 130 can provide flow-rate proportional gas samples to the mixing chamber(s) from each line. The mixing chamber(s) 110 can be sized to contain breath samples from a same phase (inhalation or exhalation) of multiple breaths, so that the mixed gas concentration measurable in the mixing chamber represents an average (for an inhalation phase or exhalation phase) across several breaths. For example, a volume of gas provided to the mixing chamber 110 from the exhalation line 130 during an exhalation phase of a breath cycle of the subject 104 is a fraction less than approximately one-half the volume of the mixing chamber 110 (e.g., a fraction from one-half to one-tenth a volume of the mixing chamber). The physical volumetric averaging by mixing samples of exhalations in the mixing chamber(s) 110 over multiple breaths corresponds to mathematical averaging and reduces and slows time variations in relative gas concentrations. This reduction and slowing in the relative gas concentrations can enable the use of slower and less expensive gas sensors to determine O₂ and CO₂_ concentrations and reduce sensitivity to T₉₀ time constants between the CO₂ and O₂ gas sensors. “T₉₀” is the time for the sensor to reach 90% of the actual gas concentration. Accordingly, slower, smaller, and less expensive O₂ and CO₂ sensors can be used in the respiratory monitoring system 100.

The CO₂ sensor(s) 115 is (are) coupled to the mixing chamber(s) 110 to detect the concentration of CO₂ gas within the mixing chamber. The CO₂ sensor(s) 115 can also be communicatively coupled to the system controller 150 to output signals indicative of CO₂ concentration to the system controller 150. Similarly, the O₂ sensor(s) 118 can be communicatively coupled to the system controller 150 to output signals indicative of O₂ concentration to the system controller 150. The O₂ sensor(s) 118 can be a commercially-available paramagnetic O₂ sensor, such as Hummingbird Sensor Technology's commercially-available Paracube® Modus oxygen sensor, though other O₂ sensors can be used. Preferably, the O₂ sensor can accurately sense the range of O₂ concentrations on the inhalation line 120, which can be as high as 100% A variety of CO₂ sensors can be employed such as single channel or dual channel non-dispersive infrared (NDIR) sensors and need only support maximum CO₂ concentrations of 10% to 15%. The CO₂ sensor(s) 115 can be, for example, a Gas Sensing Solution's SprintIR-6S CO₂ sensor with 20% CO₂ gas concentration range.

The relative humidity sensor(s) 116 is (are) used to detect the water vapor content in exhaled gas and/or inhaled gas in the mixing chamber(s). Similarly, the temperature sensor(s) 117 is (are) used to detect temperature of the exhaled gas and/or inhaled gas in the mixing chamber(s) 110. Relative humidity and temperature values of gases are used to convert measured O₂ and CO₂ concentrations to dry (no water vapor), standard temperature (0 C) and pressure (760 mm Hg) conditions (STPD conditions) when computing VO₂ and VCO₂ values, improving the accuracy of the computed values.

The first flow sensor 125 is arranged to sense a flow rate of gas in the inhalation line 120 and can be communicatively coupled to the system controller 150. The second flow sensor 135 is arranged to sense a flow rate of gas in the exhalation line 130 and also can be communicatively coupled to the system controller 150. By monitoring flow rates of inhaled and exhaled gases, inhale duration, exhale duration, CO₂ concentration, and O₂ concentration on inhaled and exhaled gas, VO₂ and can be determined (e.g., computed by the system controller 150). The system controller can be implemented as a microprocessor, microcontroller, programmable logic controller, field-programmable gate array, digital signal processor, logic circuitry, or some combination thereof.

### 2. Example Respiratory Monitoring System

FIG. 2 depicts a more detailed example of a respiratory monitoring system 100 that includes two mixing chambers 110-1, 110-2 that can be swapped between inhalation line 120 and exhalation line 130. Each mixing chamber has an input port 111 and an output port 119 to couple to valves in the gas flow circuits. To accomplish line swapping, the inhalation flow circuit 128 and exhalation flow circuit 138 (together referred to as sampling flow circuit 233) comprise multiple flow lines, a four-way valve 280 (which may be referred to as a cross-over valve), two three-way valves 211, 212, and two, 2:1 flow-line couplers 229, 239. Two of the flow lines in the inhalation flow circuit 128 connect to two flow-rate proportional, passive flow dividers 224, 226 that are coupled to the inhalation line 120. A first input-sampling flow line 241 connects to the four-way valve 280 and the output flow line 242 connects to one of the 2:1 flow-line couplers 229. Two of the flow lines in the exhalation flow circuit 138 connect to two additional flow-rate proportional, passive flow dividers 234, 236 that are coupled to the exhalation line 130. A second input-sampling flow line 243 connects to the four-way valve 280 and the output flow line 244 connects to the second of the 2:1 flow-line couplers 239. Two ports of the four-way valve 280 connect to input ports of the two mixing chambers 110-1, 110-2. The four-way valve 280 enables switching of the input-sampling flow lines 241, 243 to inputs of each of the two mixing chambers 110-1, 110-2, alternating connection of the inputs of the mixing chambers to either the inhalation line 120 or the exhalation line 130. The two, 2:1 flow-line couplers 229, 239 and two three-way valves 211, 212 are connected with flow lines such that there is a cross-connect between exhaust ports of the two mixing chambers 110-1, 110-2.

The sampling of air from and return of air to the inhalation line 120 and the sampling of air from and return of air to the exhalation line 130 can be located away from the subject's Y-piece 107. Therefore, the sampling apparatus can be installed in a way that does not impact the Y-piece 107 or other instrumentation such as a capnometer that may be used to monitor the subject's breathing.

In some implementations, the four-way valve 280 and the two three-way valves 211, 212 are manually operated. In other implementations, one or all of these valves are controlled via the system controller 150. For example, the valves may be solenoid type valves that can be toggled with command signals issued by the system controller 150. In such cases, relays may be used to convert signals from the system controller 150 into control signals with sufficient power to operate the valves.

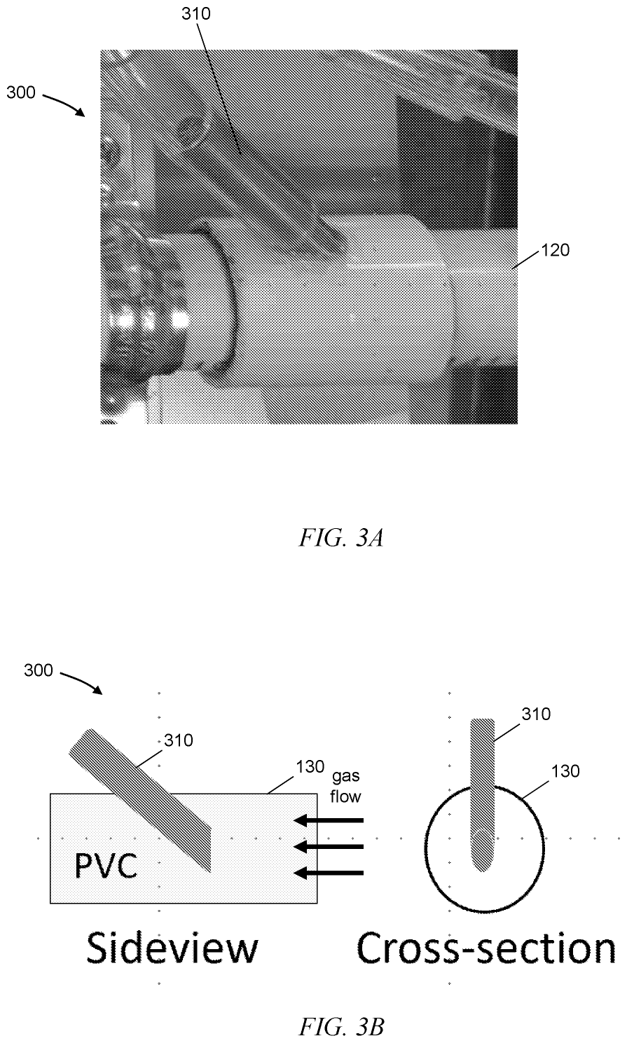

The passive flow dividers 224, 226, 234, 236 are configured for flow-rate proportional, side-stream sampling of the gas passing through the inhalation line 120 and exhalation line 130. FIG. 3A and FIG. 3B depicts an example of such a passive flow divider 300. Each passive flow divider that is used to sample a gas (inhalation or exhalation) can provide the sampled gas to its coupled mixing chamber at a flow rate that is proportional to a flow rate of gas in the line (inhalation or exhalation) from which it samples the gas. The passive flow divider 300 includes an inner flow-divider pickoff tube 310 that inserts into the larger-diameter inhalation line 120 or exhalation line 130. Because the diameter of the flow-divider pickoff tube 310 can be significantly smaller than the diameter of the line into which it inserts, the flow-rate proportional gas sampling can be carried out without imposing a significant respiratory burden on the patient while delivering rate-proportional fractions of each breath to a miniature mixing chamber 110 that is 50 to 100 times smaller than a full breath mixing chamber.

The passive flow divider 300 reduces inaccuracies associated with constant-flow sampling of the gas. During each exhaled breath, gas concentrations and flow rate are dynamic, changing in time over the course of the exhalation. A side-stream sampling system that employs a constant-rate pump to sample the exhaled breath would not preserve the average gas concentrations over a breath. Instead, the constant pump sampling weights the end-tidal concentrations too heavily and the peak exhalation concentrations too weakly. The passive flow divider 300 can preserve, in the side-stream samples delivered to the mixing chamber(s) 110, the time variations in flow rate and gas concentrations for each exhalation, weighting end-tidal concentrations and peak concentrations accurately.

The passive flow dividers 224, 226, 234, 236 enable passive, flow-rate proportional side-stream sampling of the main flows in the inhalation line 120 and exhalation line 130. The proportion of mainstream flow diverted to the appropriate mixing chamber 110-1, 110-2 is set in part by the cross-sectional area of the flow-divider pickoff tube 310 relative to the cross-sectional area of the inhalation or exhalation line into which the flow-divider pickoff tube 310 is inserted. In some cases, the passive flow dividers 224, 226, 234, 236 can be located to take advantage of the differential pressure drop across the flow sensors 125, 135. For example, an input flow line 241 to a mixing chamber 110-1 can be located upstream of a flow sensor 125 and the return flow line 242 from the mixing chamber 110-1 can be located downstream from the flow sensor 125, as illustrated in FIG. 2. Performing flow-rate proportional side-stream sampling of the gas mixtures in the inhalation line 120 and exhalation line 130 can allow the use of miniature mixing chambers 110-1, 110-2 with low-cost, slow gas sensors (ten second or longer Too response times) and also make the respiratory monitoring system 100 agnostic to the mechanical ventilator 170 or other gas source to which it may connect. For example, the respiratory monitoring system 100 can operate without information regarding gas flow or gas concentration being provided by the mechanical ventilator 170 or other gas source.

The two mixing chambers 110-1, 110-2 can have a same size and same CO₂, O₂, relative humidity, and temperature sensors coupled to or within each mixing chamber to detect their respective gas parameters. Additional sensors, such as sensors for specific gases, molecules, and/or compounds, can also be coupled to each mixing chamber 110-1, 110-2 in some applications. The mixing chambers can be small in size (e.g., each having a volume no larger than 100 ml or even no larger than 50 ml) so that the two mixing chambers 110-1, 110-2, flow circuitry, sensors, and system controller 150 can be packaged in a unit with a small form factor (e.g., occupying a volume less than 0.5 cubic feet).

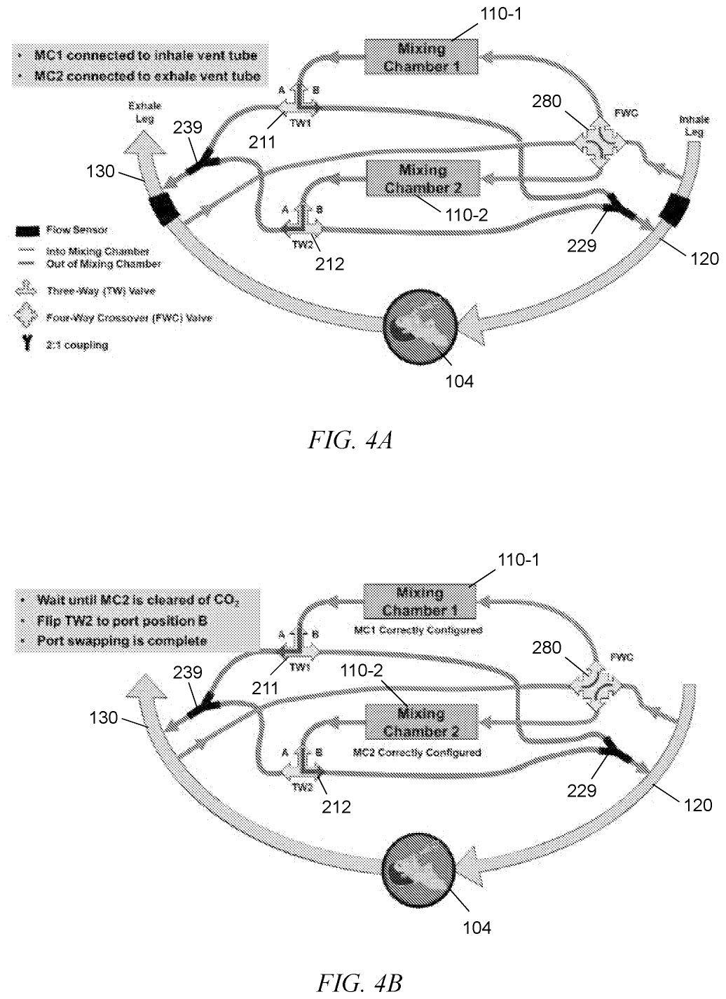

FIG. 4A depicts the respiratory monitoring system 100 of FIG. 2 operating with a first valve configuration. The four-way valve 280, and two three-way valves 211, 212 are toggled such that gas sampled from the inhalation line 120 flows into the first mixing chamber 110-1 and returns to the inhalation line 120 via the first three-way valve 211 and the first 2:1 flow-line coupler 229. Additionally, gas from the exhalation line 130 flows into the second mixing chamber 110-2 via the four-way coupler 280 and flows from the second mixing chamber to the exhalation line 130 via the second three-way valve 212 and the second 2:1 flow-line coupler 239.

During a first interval of time, when the respiratory monitoring system 100 has the valve configuration of FIG. 4A, the first mixing chamber 110-1 can receive inhalation gas for the subject 104. The inhalation gas can be sampled from an inhalation line 120. A first concentration of oxygen and carbon-dioxide in the first mixing chamber 110-1 can be detected with a first oxygen sensor and a first carbon-dioxide sensor in the first mixing chamber 110-1 during the first interval of time. When the subject exhales, during a second interval of time and with the system in the same valve configuration, the second mixing chamber 110-2 can receive exhalation gas from the subject. The exhalation gas is sampled from the exhalation line 130. A second oxygen sensor and second carbon-dioxide sensor coupled to the second mixing chamber 110-2 can detect a second concentration of oxygen and carbon-dioxide in the second mixing chamber 110-2 with the second oxygen sensor and second carbon-dioxide sensor during the second interval of time.

FIG. 4B depicts the respiratory monitoring system of FIG. 2 operating with a second valve configuration that swaps the mixing chambers between inhalation and exhalation lines. In this valve configuration, the gas from the inhalation line 120 flows into the second mixing chamber 110-2 via the four-way valve 280 and then back to the inhalation line via the second three-way valve 212 and first 2:1 flow-line coupler 229. Gas from the exhalation line 130 flows into the first mixing chamber 110-1 via the four-way valve 280 and then back to the exhalation line via the first three-way valve 211 and second 2:1 flow-line coupler 239.

During a third interval of time, when the respiratory monitoring system 100 has the valve configuration of FIG. 4B, the second mixing chamber 110-2 can receive inhalation gas for the subject 104. The inhalation gas received by the second mixing chamber 110-2 can be sampled at a same location along the inhalation line 120 that was used for the first mixing chamber 110-2. A third concentration of oxygen and carbon-dioxide in the second mixing chamber 110-2 can be detected with the second oxygen sensor and second carbon-dioxide sensor in the second mixing chamber 110-2 during the third interval of time. When the subject exhales, during a fourth interval of time and with the system in the same valve configuration of FIG. 4B, the first mixing chamber 110-1 can receive exhalation gas from the subject. The exhalation gas can also be sampled at the same location along the exhalation line 130. The first oxygen sensor and first carbon-dioxide sensor can detect a fourth concentration of oxygen and carbon-dioxide in the first mixing chamber 110-1 during the fourth interval of time. The detected first, second, third, and fourth concentrations of oxygen can be used as described herein to compute a value relative to the health of the patient such as, but not limited to, oxygen consumption, calories burned, macronutrient utilization, etc.

When switching from the configuration of FIG. 4A to the valve configuration of FIG. 4B, toggling of the second three-way valve 212 can be delayed to clear CO₂ from the second mixing chamber 110-2. For example, the delay in toggling can provide at least one-half the volume of gas in the second mixing chamber 110-2 to the exhalation line 130 before changing, with the three-way valve 212, from coupling the output port of the second mixing chamber 110-2 to the exhalation line 130 to coupling the output port of the second mixing chamber 110-2 to the inhalation line 120. In some cases, the delay may be several respiratory cycles. In some implementations, the delay in toggling can be based on a measured concentration of CO₂ in second mixing chamber 110-2. For example, the toggling can be delayed until the measured concentration of CO₂ reduces to a predetermined value or lower, such as 1% or lower, 0.5% or lower, 0.2% or lower, or even 0.1% or lower. Gas-concentration measurements may be suspended during the purging of CO₂ from the mixing chamber. A similar delay can be used for toggling the first three-way valve 211 when switching from the configuration of FIG. 4B to the valve configuration of FIG. 4A.

The inclusion of mixing chambers 110-1, 110-2 and the passive flow-rate proportional sampling allows the use of slow, relatively low-cost gas sensors. Additionally, the system can sample gases without using an active gas control system to ensure preservation of the exhaled gas mixture percentages. Another aspect of the architecture can be the use of identical mixing chambers for both the inhalation line 120 and the exhalation line 130 with the capability to swap the mixing chambers as described above. This chamber-swapping feature can, for example, permit cross-checking calibration (e.g., comparing measurement results) of the O₂ and CO₂ gas sensors coupled to each mixing chamber without disrupting the normal operation of a mechanical ventilator 170 that may be connected to the respiratory monitoring system 100. For example, normal CO₂ concentration in the inhalation line 120 is approximately 0.04% or less so a larger number would indicate a drift in the zero point for the CO₂ sensor in the mixing chamber 110 connected to receive a sample of inhalation gas from the inhalation line 120. Additionally, for a fixed O₂ setting on the ventilator, the O₂ concentration measured on samples collected from the inhalation line 120 should be the same for the O₂ sensors in either mixing chamber. Differences in the inhale O₂ concentration observed when the mixing chambers are swapped imply a sensor error (e.g., gain error) in one or both of the O₂ sensors. Repeated swaps of the mixing chambers 110-1, 110-2 can be made to confirm the sensor error. In addition, by chamber swapping, it is possible to collect inhale and exhale data using a single, same mixing chamber, as described below. When volumetric gas averaging is done, the swapping can occur after a number of respiratory cycles (e.g., from three to 20 respiratory cycles) to allow sufficient averaging before swapping from exhalation gas to inhalation or vice versa. By using one mixing chamber, additive bias terms that might appear over time in the O₂ gas sensor and/or CO₂ gas sensor calibration(s) can be automatically cancelled and the impact of gain errors can be reduced. For example, an O₂ sensor having a bias that gives an artificially high O₂ concentration on inhaled gas would give an equally high O₂ concentration on exhaled gas. The error would cancel when taking the difference in concentrations between the inhaled and exhaled gases.

The chamber swapping on each of the inhalation line 120 and exhalation line 130 can be performed multiple times during system use and at regular time intervals (e.g., every 30 seconds, every minute, every five minutes, or other time interval). The time period between mixing chamber swaps can be long enough to ensure the mixing chamber is purged of old gas and has reached a steady state, which may take more than one minute for some implementations (e.g., from 90 seconds to 6 minutes). Latencies on the order of 10 minutes or longer between mixing chamber swaps can increase the possibility that changes in the patient metabolism between the inhale and/or exhale comparisons collected with each chamber may bias the resulting computation of VO₂ consumption. The swapping rate can depend on the volume of the mixing chambers (which determines a number of samples averages and an amount of gas used to purge the mixing chamber), speed of the gas sensors, respiratory rate of the patient, and breath volume of the patient. In some cases, the swapping period can be determined based on a number of respiratory cycles of the patient (e.g., a number of respiratory cycles from 10 to 200 are detected by the flow sensor before swapping chambers). There are advantages to using two mixing chambers 110-1, 110-2 in a respiratory monitoring system 100 and implementing chamber swapping on the inhalation line 120 and exhalation line 130. These advantages include:
Random Error Reduction: By swapping mixing chambers 110-1, 110-2, the gas measurements from the two mixing chambers for each of the inhalation line 120 and exhalation line 130 can be averaged to reduce random error. For example, VO₂ for a patient can be computed as follows:

$${VO}_{2} = \frac{1}{2} ( {VO}_{2 , {MC}_{1}} + {VO}_{2 , {MC}_{2}} ) \qquad ( 3 )$$

where VO_{2,MC}_{₁} is determined using the first mixing chamber 110-1 and VO_{2,MC}_{₂} is determined using the second mixing chamber 110-2 (e.g., determined using EQ. 4 below).
Calibration Checks: The chamber swapping, without changing ventilator settings, can be used to confirm consistency of measurements. With chamber swapping, the respiratory monitoring system 100 should produce two (theoretically equivalent) VO₂ measurements, for example. One measurement obtained using the first mixing chamber 110-1 and its O₂ sensor should agree with a second measurement obtained using the other mixing chamber 110-2) and its O₂ sensor. Differences between the measurements with both chambers on one line can be monitored to detect a possible degradation in calibration. This calibration check can be accomplished without disruption of patient ventilation, for example.
Redundancy: If sensor drift in one chamber occurs by more than a threshold amount or its sensor fails, its data can be ignored and the other mixing chamber can be used until the sensor is recalibrated or serviced. Note that recalibration and sensor servicing for a mixing chamber can be possible without disrupting patient ventilation. For example, respiratory monitoring can continue with one mixing chamber by including provision for a bypass circuit around the other mixing chamber while the other mixing chamber is replaced or otherwise serviced.
Cancellation of Bias in the Gas Sensors: A frequent form of paramagnetic O₂ sensor error and CO₂ sensor error is a zero offset or bias. If the mechanical ventilator 170 settings are only changed occasionally, a potential method of cancelling the bias term is to use the gas concentrations from the same mixing chamber 110-1 to calculate VO₂ and VCO₂, for example, measured on the inhalation line 120 and exhalation line 130 at two consecutive times to compute the differential volume fractions of gas. This measurement approach is termed common-mode rejection. The use of separate sensors (e.g., O₂ sensors on the inhalation line 120 and exhalation line 130 to determine VO₂) without common-mode rejection can result in errors from both sensors adding to increase the overall system error.
Reduction of Bias in the Flow Sensors: Using the Haldane transform, the volume changes in O₂ and CO₂ can be referenced to either the inhalation flow sensor 125 or the exhalation flow sensor 135 and whichever flow sensor is more accurate will provide a more accurate measure of VO₂ and VCO₂. As with the gas calibration check, differences in the VO₂ and VCO₂ attributable to whichever flow sensor is referenced provide an indication of bias between the two flow sensors.
Tracking CO₂ and O₂ Gas Sensor Calibration Drift: By calibrating the sensors in the two mixing chambers 110-1, 110-2 and subsequently checking the calibrations over time with certified gases, insight can be gained into the calibration stability of the gas sensors and also whether calibration errors are due to individual sensor drift or, if correlated across the sensors, are more likely indicative of an error in the calibration procedure. This calibration information is useful in the prototyping and manufacturing phases to validate the performance of the selected gas sensors, identify alternative sensors if needed, and for quality assurance.
Indication of the Source of Errors Based on Ventilator Output: Without being bound to a particular theory, with a metabolic sensor on the inhalation line 120 as well as the exhalation line 130 (assuming the gas volume fractions and flow measurements are highly accurate and the gases are well mixed), the values for VO₂ and VCO₂ can be calculated generally as the exhale gas volume fraction, Fₑ, times the exhale volumetric flow, Qₑ, on the exhalation line 130 minus the inhale gas volume fraction, Fᵢ, times the volumetric flow, Qᵢ, on the inhalation line 120 as follows:

$${VO}_{2} = {F}_{e , {O}_{2}} {Q}_{e} - {F}_{i , {O}_{2}} {Q}_{i} \qquad ( 4 )$$

$${VCO}_{2} = {F}_{e , {CO}_{2}} {Q}_{e} - {F}_{i , {CO}_{2}} {Q}_{i} \qquad ( 5 )$$

Again, the values VO₂ and VCO₂ can be expressed in LPM and the gas concentrations converted to STPD conditions. Employing a ventilator with known volume rates of O₂ injected, computing VO₂ and VCO₂ using EQ. 4 and EQ. 5 or using other equations to compute these values, assuming no gas is lost out of the system (conservation of gas), and comparing the computed flows based on measurements with the known injection rate of O₂ from the ventilator provides an indication of the cumulative error arising from inaccuracy of the gas sensors combined with inaccuracy of the flow sensors. Detecting errors in this way can be important in situations where a subject 104 is on a mechanical ventilator 170 for days or weeks, since calibration might drift over these periods of time. Such errors would not otherwise be detectable without disrupting the patient ventilation.

Computer control of the four-way valve 280 allows chamber-swapping to be executed by an automated machine process according to specified protocols. For example, a protocol may be implemented to automatically provide periodic calibration checks and/or to cancel or reduce the effects of bias in either or both of the gas or flow measurements.

For the respiratory monitoring system 100 described above in connection with FIG. 2, two three-way valves 211, 212 and two flow line couplers 229, 239 are used (instead of a another four-way coupler) to alternatingly couple each output from the two-mixing chambers 110-1, 110-2 to either the inhalation line 120 or the exhalation line 130. The arrangement of three-way valves 211, 212 and flow line couplers 229, 239 allows independent switching of the mixing chamber outputs to purge CO₂ from the exhalation mixing chamber. When switching the mixing chamber 110-2 on the exhalation line 130 (see FIG. 4A) to the inhalation line 120 (see FIG. 4B), switching of the output port of the exhalation mixing chamber 110-2 can be delayed until residual CO₂ in the mixing chamber 110-2 has been displaced by the inhalation gas mixture from the inhalation line 120. Accordingly, the second three-way valve 212 can remain in its previous configuration (that of FIG. 4A) until CO₂ is sufficiently purged from the mixing chamber 110-2, which has already been switched to an inhalation mixing chamber. Measurement of the CO₂ level in the mixing chamber 110-2 can be monitored to determine an appropriate time to switch the output port of the mixing chamber 110-2 to the inhalation line 120 (establishing the valve configuration in FIG. 4B). Alternatively, a pre-determined fixed delay (e.g., in terms of a fixed time or number of respiratory cycles after switching of the four-way valve 280) can be used to delay the switching of the three-way valve coupled to an output port of the exhalation mixing chamber.

The respiratory monitoring system 100 described above in connection with FIG. 2 can provide a cost-effective instrument for 24/7 tracking of energy and macronutrient utilization of mechanically ventilated patients. The instrument can be non-invasive or non-disrupting to a mechanically-ventilated patient. The measured data throughout each day can be used to tailor nutrition for each patient. Replacing current parametric estimators of nutritional feeding regimens with nutrition reflecting actual energy expenditure and macronutrient utilization measured by indirect calorimetry can improve both patient recovery time and patient mortality.

In some implementations, the respiratory monitoring system 100 can be used for VO₂ measurements only, with VCO₂ (or other gas) measurements obtained by other means such as, but not limited to, mainstream capnography for which a capnometer can be inserted in the patient line 520.

### 3. Gas Sampling

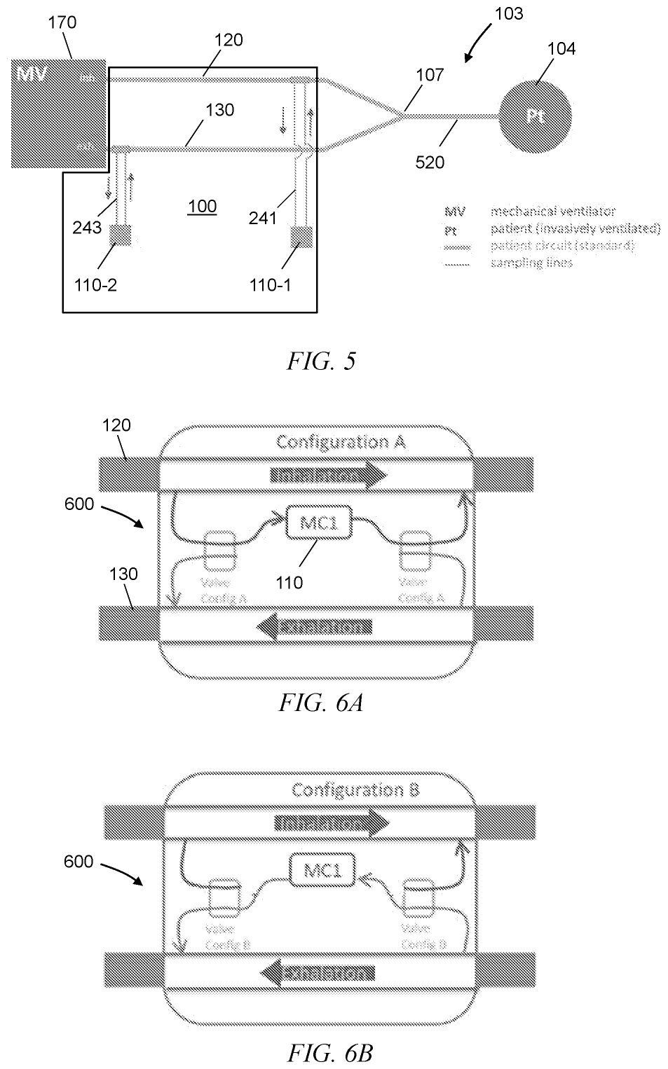

To further improve measurement accuracy, at least the location at which exhalation gas is sampled is chosen judiciously. FIG. 5 depicts an implementation for a respiratory monitoring system 100 in a clinical setting where a subject 104 is ventilated by a mechanical ventilator 170. The input-sampling line 243 for the exhaled gas connects to the exhalation line 130 as far as possible from the patient 104. For example, the input-sampling line 243 may connect to the exhalation line 130 at a location that is within 6 inches from a distal end of the exhalation line 130 near or at the mechanical ventilator 170. Distance from the patient 104 (and from the subject's Y-piece 107 if present) can provide for mixing of the exhaled gas with any residual inhalation gas in the dead space of the patient line 520 before the gas mixture is sampled. The subject's Y-piece 107 and patient line 520 can be part of the breathing apparatus 103 for the subject 104. In some cases, the distance of the sampling location can be no smaller than any distance in a range from 2 feet to 10 feet from the patient. Using an exhalation line 130 with ribbing and/or interior protruding features can aid in mixing the exhalation gas before it is sampled.

Allowing substantial mixing of the exhalation gas after the subject's Y-piece 107 and before sampling is advantageous instead of sampling at the subject's Y-piece 107. The concentration of exhalation gas at the subject's Y-piece 107 can vary abruptly as gas from the respiratory dead space in the patient line 520 (nominally with the same O₂ concentration as inhalation gas) is followed by gas coming from the patient's alveoli, where gas exchange occurs and the reduction in O₂ concentration is higher. By sampling the exhalation gas a substantial distance (e.g., at least 12 inches, at least 24 inches or more) after any dead space in the patient line 520, the transition from higher O₂ concentration from the dead space to lower concentration from the alveoli becomes smoother. The advantage is twofold: (1) additional pre-mixing of gas to enhance the physical averaging of the mixing chambers and (2) smoother variation of O₂ (and other gases, including CO₂) concentration, to reduce the negative impact of imperfect proportional sampling.

The mixing advantage is not as significant for inhalation gas, since ventilators are designed to provide inhalation gas with nominally constant concentrations. However, when the inhaled oxygen fraction (FiO₂) is higher than ambient, the gas mixing controller of the ventilator is never perfect (standards allow for +/−2.5% variation of O₂ concentration from the nominal value set by the clinician). To provide respiratory exchange ratios that are accurate to within 10%, the absolute error in measuring O₂ and CO₂ concentrations are less than or equal to 0.2% absolute and a ±2.5% error is 12.5 times the allowable error. Locating the inhale and exhale passive flow dividers 224, 236 to provide thorough mixing helps to reduce errors in measurement.

Similarly, the input-sampling line 241 for the inhaled gas can connect to the inhalation line 120 as close as possible to the patient 104. In the illustration of FIG. 5, the input-sampling line 241 located close to the subject's Y-piece 107 (e.g., within 6 inches of the Y-piece) that couples the inhalation line 120 and exhalation line 130 to the patient line 520. In some cases, the input-sampling line 241 can be located near the patient's mouth (e.g., within 6 inches of the patient's mouth) in the patient line 520. In some implementations, the input-sampling line 241 for the inhaled gas can connect to the inhalation line 120 anywhere along the inhalation line since the inhalation gas is unlikely to significantly change in composition over time.

As mentioned above, the mixing chambers 110-1, 110-2 can provide a physical averaging of sampled gas over time (e.g., across 2 to 10 breaths or more). Flow rates measured by the flow sensors 125, 135 can also be averaged across the same number of breaths. Accordingly, both the measured gas concentration (e.g., exhaled O₂ concentration) and flow rate can be slowly varying quantities (varying over time scales on the order of one second or more). As such, there is no tight time requirement (e.g., high synchronicity) for multiplying the measured concentration and flow rate to obtain VO₂ values, as there would be in a mixing-chamber free instrument that uses higher-frequency measurements of gas concentration and flow. Since delays between the measured gas concentration and flow rate can vary over time (e.g., with changes in the ventilator settings and/or breathing circuit hardware), avoiding strict synchronization of measurements for computation of VO₂ is another advantage of having a mixing chamber.

Additionally, the slow variations in measured gas concentrations allows the use of slower, less expensive, gas sensors. Normally, a mixing chamber 110-2 is used only for expired gas because the gas concentration of expired gas can change quickly (on time scales less than one second) throughout the exhalation phase, whereas it is theoretically constant throughout the inhalation phase. Most ventilators do not control the O₂ concentration of inspired gas tightly. For example, typical standards for ventilators is to deliver O₂ concentration in the inspired gas within +2.5% from the nominal value set by the clinician. The respiratory monitoring system 100 can measure FᵢO₂ via a mixing chamber (with proportional sampling). The mixing-chamber-based measurement of FᵢO₂ improves accuracy over traditional time-averaged FᵢO₂ measurements, which are equivalent to constant-flow sampling.

For the concentration of the sampled gas in a mixing chamber to be the same as the concentration of mixed gas from the whole breath, sampling preferably should be proportional to the flow rate in the inhalation line 120 or exhalation line 130 as opposed to constant-flow sampling. In such flow-rate proportional sampling, the instantaneous flow to the mixing chamber needs to be a fixed percentage of the instantaneous gas flow in the inhalation or exhalation line which is being sampled. The passive flow divider 300 (shown in FIG. 3A) can be used for each of the passive flow dividers in the respirator monitoring system 100 and provides such flow-rate proportional gas sampling. Passive flow dividers 300, 226, 234 when coupled to output ports from the mixing chambers 110-1, 110-2 operate as flow combiners to reintroduce gas into the inhalation line 120 or exhalation line 130. Instantaneous flow-rate proportional sampling includes averaging (due to physical, instrument limitations) over periods of time that are short compared to the entire duration of a breath. Constant-flow sampling would generate gas concentrations in the mixing chamber that would be biased towards the concentration that the gas has at lower flow rates within a breath (e.g., towards the end of exhalation as opposed to the when the flow peaks shortly after the beginning of exhalation.

Another feature of the gas sampling is that a small fraction of the total gas flowing in the inhalation line 120 and exhalation line 130 is sampled (using a flow-rate proportional paradigm). The small amounts of sampled gases allow the mixing chambers to be smaller and the respiratory monitoring system 100 to be more compact.

### 4. Single Mixing Chamber Implementation

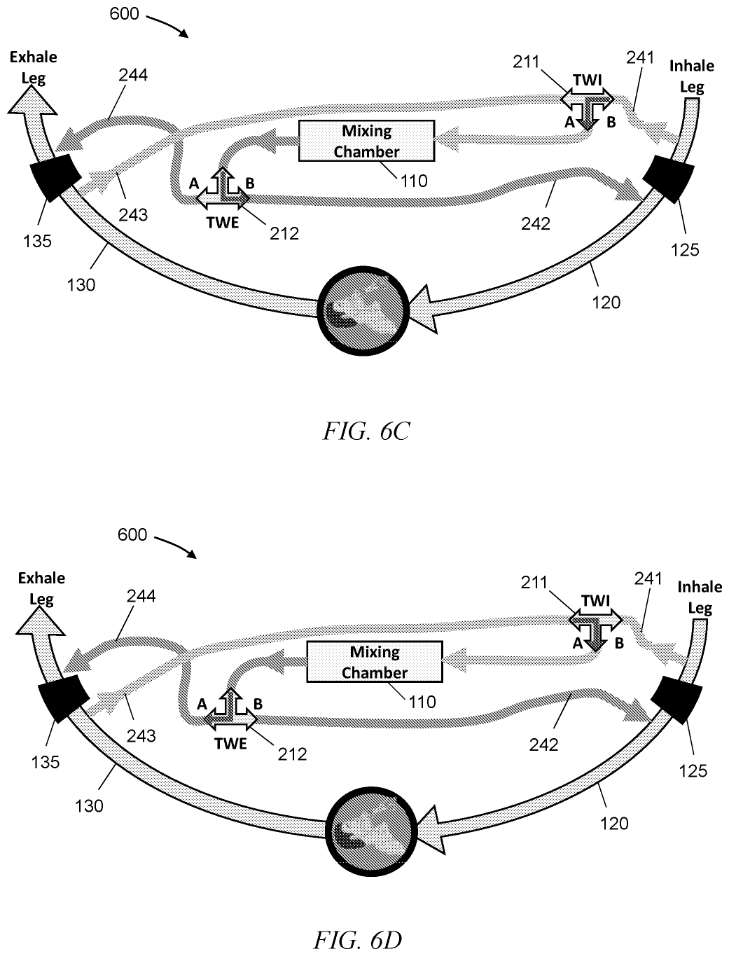

FIG. 6A and FIG. 6B are block diagrams for an implementation of a respiratory monitoring system 600 that uses a single mixing chamber 110. The mixing chamber 110 can be switched with valves to sample and monitor gas on the inhalation line 120 for a period of time, as depicted in FIG. 6A. The mixing chamber 110 can also be switched with the valves to sample and monitor gas on the exhalation line 130 for a period of time, as depicted in FIG. 6B. The switching can be periodic as described above for the two-mixing-chamber implementation. Because the same gas sensors are used to monitor inhalation gas and exhalation gas, some of the benefits described above (such as common-mode rejection of error) for the two-mixing-chamber system also apply to the single-mixing-chamber system.

The flow line and valve arrangement for the respiratory monitoring system 600 of FIG. 6A and FIG. 6B can be understood from FIG. 6C and FIG. 6D. The respiratory monitoring system 600 can be implemented using one mixing chamber 110 two three-way valves 211, 212, and flow lines connected between the components as depicted in FIG. 6C. The flow lines connected to the ports of the first three-way valve 211 can couple to passive flow dividers in the inhalation line 120 and exhalation line 130 and to an input port of the mixing chamber 110. This first three-way valve 211 can switch gas sampling inputs to the mixing chamber 110 between the inhalation line 120 and exhalation line 130. The second three-way valve 212 has flow lines connected to its ports such that it can switch gas outputs from the mixing chamber 110 to be delivered to passive flow dividers (used as combiners) in the inhalation line 120 or the exhalation line 130. The three-way valve configuration in FIG. 6C corresponds to the respiratory monitoring system 600 arrangement of FIG. 6A. The three-way valve configuration in FIG. 6D corresponds to the respiratory monitoring system 600 arrangement of FIG. 6B.

### 5. Estimating VO₂ and Energy Expenditure

Without being bound to a particular theory, VO₂ can be estimated for a subject using the respiratory monitoring system 100 of FIG. 2 and the following equation:

$${VO}_{2} = \frac{1}{2} [ {Q}_{e} ( {F}_{i , {O}_{2} , 1}^{A} \frac{1 - {F}_{e , {O}_{2} , 1}^{B} - {F}_{e , {CO}_{2} , 1}^{B}}{1 - {F}_{i , {O}_{2} , 1}^{A} - {F}_{i , {CO}_{2} , 1}^{A}} - {F}_{e , {O}_{2} , 1}^{B} ) + {Q}_{e} ( {F}_{i , {O}_{2} , 2}^{B} \frac{1 - {F}_{e , {O}_{2} , 2}^{A} - {F}_{e , {CO}_{2} , 2}^{A}}{1 - {F}_{i , {O}_{2} , 2}^{B} - {F}_{i , {CO}_{2} , 2}^{B}} - {F}_{e , {O}_{2} , 2}^{B} ) ] , \qquad ( 6 )$$

where F represents a measured fractional content of the gas, Qₑ represents an average flow rate of the gas in the exhalation line 130, the first subscript for F (“i” or “e”) represents inhalation or exhalation, respectively, the second subscript for F identifies the type of measured gas concentration, the third subscript for F (“1” or “2”) identifies which mixing chamber (first or second) at which the gas was measured, and the superscript for F (“A” or “B”) identifies which valve configuration the respiratory monitoring system 100 is in when the gas measurement is made. The “A” configuration is when the first mixing chamber (1) receives gas samples from the inhalation line 120 and the second mixing chamber (2) receives gas samples from the exhalation line 130, as depicted in FIG. 4A. The “B” configuration is when the first mixing chamber (1) receives gas samples from the exhalation line 130 and the second mixing chamber (2) receives gas samples from the inhalation line 120, as depicted in FIG. 4B.

EQ. 6 makes use of the Haldane transform or Haldane transformation, which is based on the assumption that the nitrogen (N₂) fractions in the inhaled and exhaled air are conserved (1−FᵢO₂−FᵢCO₂=1−FₑO₂−FₑCO₂ assuming the gas concentrations are corrected for water vapor and expressed in STPD units). As described above, the measured gas concentrations can be converted to dry STPD conditions using data measured by the relative humidity sensor 116 and temperature sensor 117 coupled to each mixing chamber 110-1, 110-2.

The Haldane transform provides more plausible estimates of VO₂ in some situations than in others. If the N₂ fractions in the inhaled and exhaled air are not conserved, for example, the Haldane transform may provide less plausible estimates. Similarly, at a fractional oxygen content of 80% or higher

$$( {F}_{i , {O}_{2} , 1}^{A , B} \geq 0.8 ) ,$$

then the Haldane transform becomes less credible. Indeed, at a fractional oxygen content of

$$100 \% ( {F}_{i , {O}_{2} , 1}^{A , B} = 1 ) - e . g . ,$$

if the subject is inhaling 100% O₂ from a ventilator-then the fractions in the Eq. 6 become undefined. In these situations, the Eschenbacher transformation may provide more accurate estimates of VO₂. For more on the Eschenbacher transformation, see, e.g., Lang S, Herold R, Kraft A, Harth V, Preisser A M (2018) Spiroergometric measurements under increased inspiratory oxygen concentration (FIO2)—Putting the Haldane transformation to the test. PLOS ONE 13(12): e0207648. https://doi.org/10.1371/journal. pone.0207648, which is incorporated herein by reference in its entirety for all purposes.

The average flow rate [symbol] can be obtained by integrating the instantaneous flow rate measured by the exhalation flow sensor 135 over the duration of each respiratory cycle to obtain total tidal volume per breath and dividing by the duration of each cycle to give an average flow rate for each cycle in units of LPM, for example. Values for multiple consecutive respiratory cycles can be averaged together to obtain a longer-term average flow rate.

Without being bound to a particular theory, VO₂ can be estimated for a subject using the respiratory monitoring system 600 of FIG. 6C and the following equation:

$${VO}_{2} = {Q}_{e} ( {F}_{i , {O}_{2}}^{A} \frac{1 - {F}_{e , {O}_{2}}^{B} - {F}_{e , {CO}_{2}}^{B}}{1 - {F}_{i , {O}_{2}}^{A} - {F}_{i , {CO}_{2}}^{A}} - {F}_{e , {O}_{2}}^{B} ) , \qquad ( 7 )$$

where the third subscript for F used in EQ. 6 is dropped since there is only one mixing chamber 110. The “A” configuration is when the mixing chamber 110 receives gas samples from the inhalation line 120, as depicted in FIG. 6C. The “B” configuration is when the mixing chamber 110 receives gas samples from the exhalation line 130. EQ. 7 also makes use of the Haldane transform. Again, the measured gas concentrations can be converted to dry STPD conditions using data measured by the relative humidity sensor 116 and temperature sensor 117 coupled to the mixing chamber 110.

Once VO₂ and VCO₂ are determined, energy expenditure of the subject can be estimated. In general, VO₂ and VCO₂ are computed as average values over a breath or several breaths, obtained by using measured O₂ percent and CO₂ percent, along with the tidal volume and respiration rate, to compute a LPM volume of O₂ consumed and CO₂ produced by a subject. The LPM values can be used to compute a respiratory exchange ratio (RER) and energy expenditure via the Wier formula.

A portion of the inhalation gas that bypasses the Y-piece 107 (which can occur in a system having a ventilator operating with constant positive pressure on the inhalation line 120) contains fixed concentrations of O₂ and CO₂ on either side of the Y-piece 107 (inhalation side and exhalation side). These fixed concentrations each represent a fixed amount of O₂ and CO₂ for the portion of gas that bypasses the subject 104. These fixed amounts essentially cancel each other in the calculations used to determine VO₂ and VCO₂ (for a system having negligible leaks). This cancellation can be most easily understood with reference to EQ. 1 and EQ. 2, and also occurs with EQ. 6 and EQ. 7.

### 6. Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that inventive embodiments may be practiced otherwise than as specifically described. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein, unless clearly indicated to the contrary, should be understood to mean “at least one.”

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” shall have its ordinary meaning as used in the field of patent law.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Unless stated otherwise, the terms “approximately” and “about” are used to mean within +20% of a target dimension in some embodiments, within ±10% of a target dimension in some embodiments, within ±5% of a target dimension in some embodiments, and yet within ±2% of a target dimension in some embodiments. The terms “approximately” and “about” can include the target dimension. The term “essentially” is used to mean within ±3% of a target dimension.

In the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A respiratory monitoring system comprising:
a first multi-way valve;
a mixing chamber having an input port fluidically coupled to the first multi-way valve;
an oxygen sensor coupled to the mixing chamber;
an inhalation input-sampling flow line fluidically coupled at a first end to the first multi-way valve and to fluidically couple at a second end to an inhalation line that is fluidically coupled to breathing apparatus for a subject; and
an exhalation input-sampling flow line fluidically coupled at a first end to the first multi-way valve and to fluidically couple at a second end to an exhalation line that is fluidically coupled to the breathing apparatus for the subject,
wherein the first multi-way valve is configured to switch coupling of the input port of the mixing chamber between a first configuration where the input port is fluidically coupled to the inhalation input-sampling flow line and a second configuration where input port is fluidically coupled to the exhalation input-sampling flow line.

2. The respiratory monitoring system of claim 1, further comprising:
a second multi-way valve fluidically coupled to an output port of the mixing chamber;
a first output flow line fluidically coupled at a first end to the second multi-way valve and to fluidically couple at a second end to the inhalation line; and
a second output flow line fluidically coupled at a first end to the second multi-way valve and to fluidically couple at a second end to the exhalation line.

3. The respiratory monitoring system of claim 2, wherein the first multi-way valve is a four-way valve and the second multi-way valve is a three-way valve.

4. The respiratory monitoring system of claim 1, further comprising:
a carbon dioxide sensor coupled to the mixing chamber to measure a concentration of carbon dioxide in gas contained in the mixing chamber;
a relative humidity sensor coupled to the mixing chamber to measure a water vapor content of the gas contained in the mixing chamber; and
a temperature sensor coupled to the mixing chamber to measure a temperature of the gas contained in the mixing chamber.

5. The respiratory monitoring system of claim 1, wherein the mixing chamber is a first mixing chamber, the oxygen sensor is a first oxygen sensor, and the input port is a first input port, the respiratory monitoring system further comprising:
a second mixing chamber having a second input port fluidically coupled to the first multi-way valve; and
a second oxygen sensor coupled to the second mixing chamber,
wherein the first multi-way valve is further configured to switch coupling of the first input port between the first configuration where the first input port is fluidically coupled to the inhalation input-sampling flow line and the second input port is fluidically coupled to the exhalation input-sampling flow line and a second configuration where first input port is fluidically coupled to the exhalation input-sampling flow line and the second input port is fluidically coupled to the inhalation input-sampling flow line.

6. The respiratory monitoring system of claim 1, further comprising:
a passive flow divider coupled to the exhalation input-sampling flow line and to couple to the exhalation line, wherein the passive flow divider is configured to provide sampled gas from the exhalation line at a flow rate that is proportional to a flow rate of exhaled gas in the exhalation line.

7. The respiratory monitoring system of claim 6, wherein, during operation of the respiratory monitoring system, a volume of gas provided to the mixing chamber from the passive flow divider during an exhalation phase of a breath cycle of the subject is less than one-half the volume of the mixing chamber such that the gas in the mixing chamber sampled by the oxygen sensor represents a physical average of samples of exhalation gas from the subject over multiple respiratory cycles.

8. The respiratory monitoring system of claim 6, further comprising:
the exhalation line; and
a flow sensor to sample gas flow rate in the exhalation line,
wherein the passive flow divider is coupled to the exhalation line at a location upstream of the flow sensor and a distance no less than three feet from the subject.

9. The respiratory monitoring system of claim 8, wherein the exhalation line has interior ribbing and/or protruding features to mix gas flowing within the exhalation line.

10. The respiratory monitoring system of claim 1, further comprising:
a system controller communicatively coupled to the oxygen sensor and configured to:
determine a first concentration of oxygen in the inhalation line based at least in part on a first signal received from the oxygen sensor detecting gas received in the mixing chamber by way of the inhalation input-sampling flow line;
determine a second concentration of oxygen in the exhalation line based at least in part on a second signal received from the oxygen sensor detecting gas received in the mixing chamber by way of the exhalation input-sampling flow line; and
compute a value relevant to the health of the subject based at least in part on a difference between the first concentration of oxygen and the second concentration of oxygen.

11. The respiratory monitoring system of claim 10, wherein the value is a volume rate of oxygen consumed by the subject.

12. The respiratory monitoring system of claim 1, wherein the mixing chamber has a volume no larger than 50 ml.

13. A method of respiratory monitoring with a respiratory monitoring system, the method comprising:
during a first interval of time, receiving inhalation gas for a subject in a mixing chamber, wherein the inhalation gas is sampled from an inhalation line of the respiratory monitoring system and the inhalation line connects to breathing apparatus used by the subject;
detecting a first concentration of oxygen in the mixing chamber with an oxygen sensor during the first interval of time;
changing, with a multi-way valve, coupling of an input port of the mixing chamber from being fluidically coupled to the inhalation line to being fluidically coupled to an exhalation line of the respiratory monitoring system;
during a second interval of time, receiving exhalation gas from the subject in the mixing chamber, wherein the exhalation gas is sampled from the exhalation line and the exhalation line is coupled to the breathing apparatus;
detecting a second concentration of oxygen in the mixing chamber with the oxygen sensor during the second interval of time; and
computing a value relevant to health of the subject based at least in part on a difference between the first concentration of oxygen and the second concentration of oxygen.

14. The method of claim 13, further comprising:
coupling an output port of the mixing chamber to the inhalation line;
providing a portion of the inhalation gas received in the mixing chamber to the inhalation line during the first interval of time;
coupling the output port of the mixing chamber to the exhalation line; and
providing a portion of the exhalation gas received in the mixing chamber to the exhalation line during the second interval of time.

15. The method of claim 14, wherein an amount of exhalation gas received in the mixing chamber from the subject during an exhalation phase of a respiratory cycle of the subject is less than one-half the volume of the mixing chamber, such that the mixing chamber physically averages samples of exhalation gas from the subject over multiple respiratory cycles.

16. The method of claim 14, further comprising:
during the first interval of time, determining that a concentration of carbon dioxide gas in the mixing chamber reduces to at least a predetermined value before changing from coupling the output port of the mixing chamber to the exhalation line to coupling the output port of the mixing chamber to the inhalation line.

17. The method of claim 14, wherein the mixing chamber is a first mixing chamber, the oxygen sensor is a first oxygen sensor, the input port is a first input port, and the output port is a first output port, the method further comprising:
during a third interval of time, receiving exhalation gas from the subject in a second mixing chamber, wherein the exhalation gas is sampled from the exhalation line of the respiratory monitoring system; and
detecting a third concentration of oxygen in the second mixing chamber with a second oxygen sensor during the third interval of time,
wherein the value relevant to the health of the subject is further based in part on the third concentration of oxygen.

18. The method of claim 17, further comprising:
comparing, by a system controller, first data obtained from the first oxygen sensor to second data obtained from the second oxygen sensor to determine whether a first calibration of the first oxygen sensor or a second calibration of the second oxygen sensor has changed; and
in response to one of the first calibration or the second calibration changing by more than a threshold amount, ignoring subsequent data obtained from the first oxygen sensor or the second oxygen sensor for which the first calibration or the second calibration has changed more than the threshold amount.

19. The method of claim 18, wherein in response to both the first calibration and the second calibration changing by less than the threshold amount, the value relevant to the health of the subject is computed with an averaging computation that includes the first concentration of oxygen, the second concentration of oxygen, and the third concentration of oxygen.

20. The method of claim 17, further comprising:
maintaining respiratory monitoring of the subject with one of the first mixing chamber or the second mixing chamber; and
servicing the other of the first mixing chamber or the second mixing chamber without disrupting respiratory monitoring of the subject and without disrupting airflow in the inhalation line and exhalation line to and from the subject.
